(19) United States
(12) Patent Application Publication
(10) Pub. No.: US 2023/0274760 A1
(43) Pub. Date: Aug. 31, 2023

(54) VOICE PROCESSING DEVICE, VOICE PROCESSING METHOD, RECORDING MEDIUM, AND VOICE AUTHENTICATION SYSTEM

(71) Applicant: NEC Corporation, Minato-ku, Tokyo (JP)

(72) Inventors: Ling Guo, Tokyo (JP); Takafumi Koshinaka, Tokyo (JP)

(73) Assignee: NEC Corporation, Minato-ku, Tokyo (JP)

(21) Appl. No.: 18/016,789

(22) PCT Filed: Jul. 30, 2020
(86) PCT No.: PCT/JP2020/029248

§ 371 Date: Jan. 18, 2023

(51) Int. Cl. G10L 25/66; G10L 17/04; G10L 17/02; G10L 17/22; G10L 25/63; G10L 17/06

(52) U.S. Cl. CPC G10L 25/66; G10L 17/04; G10L 17/02; G10L 17/22; G10L 25/63; G10L 17/06

(57) ABSTRACT

A feature extraction unit (110) extracts, from input data based on an utterance of a person to be determined, a first feature of the input data using a discriminator that has performed machine learning using, as training data, voice data based on an utterance of the person to be determined in a normal state. An index value calculation unit (120) calculates an index value indicating the degree of similarity between the first feature of the input data and a second feature of the voice data based on the utterance of the person to be determined in the normal state. A state determination unit (130) determines whether the person to be determined is in the normal state or in an unusual state on the basis of the index value.

### TECHNICAL FIELD

The present disclosure relates to a voice processing device, a voice processing method, a recording medium, and a voice authentication system, and more particularly to a voice processing device, a voice processing method, a recording medium, and a voice authentication system for collating a speaker based on voice data.

### BACKGROUND ART

In a taxi company or a bus company, there is a “roll call” in which all crew members participate. An operation manager checks a health condition of a crew member by conducting a simple interview with the crew member. However, when checking a health condition of a crew member through an interview, the crew member may consciously or unconsciously lie, or over-trust or misperceive his/her health. Therefore, in order to reliably check a health condition of a crew member, related techniques have been developed. For example, PTL 1 discloses a technique for comprehensively determining a physical and mental health condition of a crew member by detecting electrocardiogram, electromyogram, eye movement, brain waves, respiration, blood pressure, perspiration, and the like using a biological sensor and a camera installed in a commercial vehicle on which the crew member rides.

### CITATION LIST

### Patent Literature

[PTL 1] WO 2020/003392 A
[PTL 2] JP 2016-201014 A
[PTL 3] JP 2015-069255 A

### SUMMARY OF INVENTION

### Technical Problem

However, in the related art described in PTL 1, it is necessary to install a biological sensor and a camera for each commercial vehicle owned by a company. Therefore, it may be avoided to adopt such a technique because the cost burden is large.

The present disclosure has been made in light of the above-described problem, and an object of the present disclosure is to provide a technology capable of easily determining a state of a person to be determined without requiring a user to conduct an interview with a person to be determined or without requiring a biological sensor.

### Solution to Problem

A voice processing device according to an aspect of the present disclosure includes: a feature extraction means configured to extract, from input data based on an utterance of a person to be determined, a feature of the input data using a discriminator that has performed machine learning using, as training data, voice data based on an utterance of the person to be determined in a normal state; an index value calculation means configured to calculate an index value indicating a degree of similarity between the feature of the input data and a feature of the voice data based on the utterance of the person to be determined in the normal state; and a state determination means configured to determine whether the person to be determined is in the normal state or in an unusual state based on the index value.

A voice processing method according to an aspect of the present disclosure includes: extracting, from input data based on an utterance of a person to be determined, a feature of the input data using a discriminator that has performed machine learning using, as training data, voice data based on an utterance of the person to be determined in a normal state; calculating an index value indicating a degree of similarity between the feature of the input data and a feature of the voice data based on the utterance of the person to be determined in the normal state; and determining whether the person to be determined is in the normal state or in an unusual state based on the index value.

A recording medium according to an aspect of the present disclosure stores a program for causing a computer to execute: extracting, from input data based on an utterance of a person to be determined, a feature of the input data using a discriminator that has performed machine learning using, as training data, voice data based on an utterance of the person to be determined in a normal state; calculating an index value indicating a degree of similarity between the feature of the input data and a feature of the voice data based on the utterance of the person to be determined in the normal state; and determining whether the person to be determined is in the normal state or in an unusual state based on the index value.

A voice authentication system according to an aspect of the present disclosure includes: the above-described voice processing device according to an aspect; and a learning device configured to train the discriminator using, as training data, the voice data based on the utterance of the person to be determined in the normal state.

### Advantageous Effects of Invention

According to an aspect of the present disclosure, it is possible to easily determine a state of a person to be determined, without requiring a user to conduct an interview with a person to be determined or without requiring a biological sensor.

### BRIEF DESCRIPTION OF DRAWINGS

FIG. 1 is a diagram schematically illustrating a configuration and an operation of a voice processing device according to a first example embodiment.

FIG. 2 is a block diagram illustrating a configuration of a voice processing device according to a second example embodiment.

FIG. 3 is a flowchart illustrating an operation of the voice processing device according to the second example embodiment.

FIG. 4 is a block diagram illustrating a configuration of a voice processing device according to a third example embodiment.

FIG. 5 is a flowchart illustrating an operation of the voice processing device according to the third example embodiment.

FIG. 6 is a diagram illustrating a hardware configuration of the voice processing device according to the second or third example embodiment.

FIG. 7 is a block diagram illustrating a configuration of a voice authentication system including the voice processing device according to the second or third example embodiment and a learning device.

### EXAMPLE EMBODIMENT

Hereinafter, some example embodiments will be described in detail with reference to the drawings.

### First Example Embodiment

(Configuration and Operation of Voice Processing Device X00 According to First Example Embodiment)

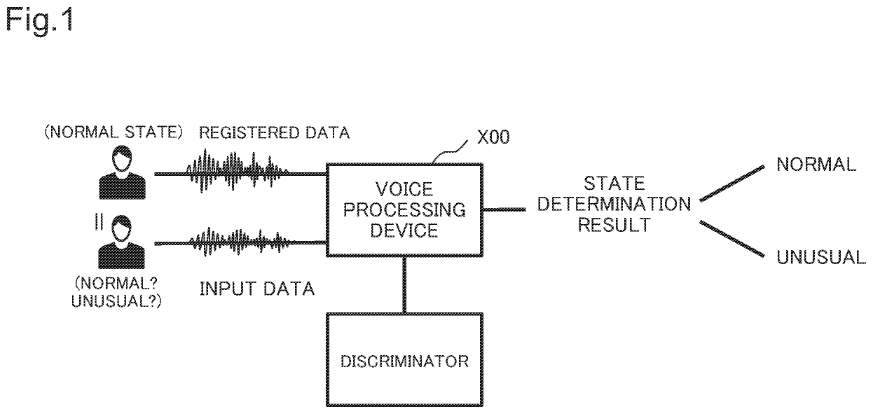

FIG. 1 is a diagram for explaining an outline of a configuration and an operation of a voice processing device X00 according to a first example embodiment. As illustrated in FIG. 1, the voice processing device X00 receives a voice signal (input data in FIG. 1) input by a person to be determined, for example, using an input device such as a microphone. An example of the person to be determined is a person whose state is to be determined by the voice processing device X00. Note that the configuration and the operation of the voice processing device X00 described in the first example embodiment can also be achieved in a voice processing device 100 according to a second example embodiment and a voice processing device 200 according to a third example embodiment to be described later.

For example, the voice processing device X00 supports a crew member (e.g., a driver) to normally perform work in a company that provides a bus operation service. In this case, the person to be determined is a crew member of a bus. Specifically, the voice processing device X00 determines a state of the crew member by a method to be described below, and decides whether the crew member can drive based on a determination result.

The voice processing device X00 communicates with a microphone installed at a specific location (e.g., a bus service office) via a wireless network, and receives a voice signal input to the microphone as input data when the person to be determined gives an utterance toward the microphone. Alternatively, the voice processing device X00 may receive, as input data, a voice signal input to a microphone worn by the person to be determined at a certain timing. For example, the voice processing device X00 receives, as input data, a voice signal input to the microphone worn by a person to be determined, immediately before the crew member, who is the person to be determined, drives a bus out of a garage.

In addition, the voice processing device X00 may receive a voice signal (registered data in FIG. 1) registered in advance in a data base (DB). The registered data is a voice signal input by the person to be determined when it is confirmed by medical examination, analysis of biological data, or the like that the person to be determined is in a normal state. The registered data is stored in the DB in association with discrimination information of the person to be determined, discrimination information of the microphone used by the person to be determined, and the like.

On the basis of the input data based on the utterance of the person to be determined and the registered data, the voice processing device X00 determines whether the person is in a normal state or in an unusual state.

In a more detailed specific example, the voice processing device X00 collates the input data based on the utterance of the person to be determined with the registered data, and determines a state of the person to be determined based on an index value indicating a degree of similarity therebetween. Here, the state of the person to be determined refers to physical and mental evaluation of the person to be determined.

In one example, the state of the person to be determined refers to a physical condition or an emotion of the person to be determined. In this case, the unusual state of the person to be determined means that the person to be determined is in a poor physical condition due to fever, insufficient sleep, or the like, the person to be determined suffers from a disease such as a cold, or the person to be determined has a psychological problem (anxiety or the like). On the other hand, the normal state of the person to be determined means that the person to be determined does not have any of the above-exemplified problems. More specifically, the normal state of the person to be determined means that the person to be determined does not have any physical or mental problem that may hinder the person to be determined from performing work or an associated duty.

Note that, in the following description, it is assumed that the person to be determined is confirmed as a person whose discrimination information has been registered together with the registered data by an operation manager through visual observation or another method. An example of another method is face authentication, iris authentication, fingerprint authentication, or another biometric authentication.

### Second Example Embodiment

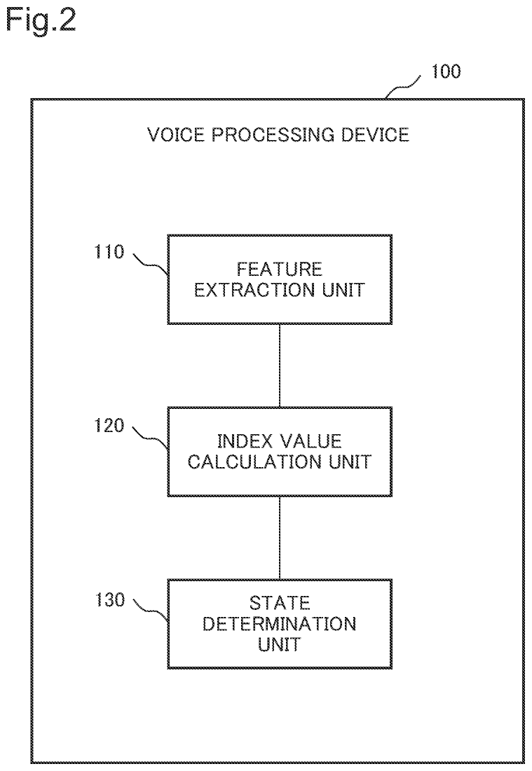

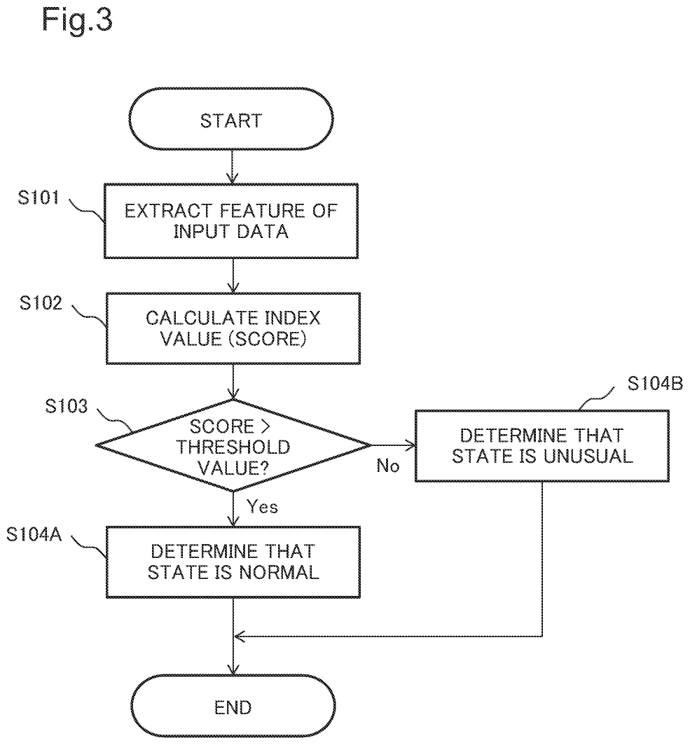

A second example embodiment will be described with reference to FIGS. 2 and 3.

(Voice Processing Device 100)

A configuration of a voice processing device 100 according to the second example embodiment will be described with reference to FIG. 2. FIG. 2 is a block diagram illustrating the configuration of the voice processing device 100.

As illustrated in FIG. 2, the voice processing device 100 includes a feature extraction unit 110, an index value calculation unit 120, and a state determination unit 130.

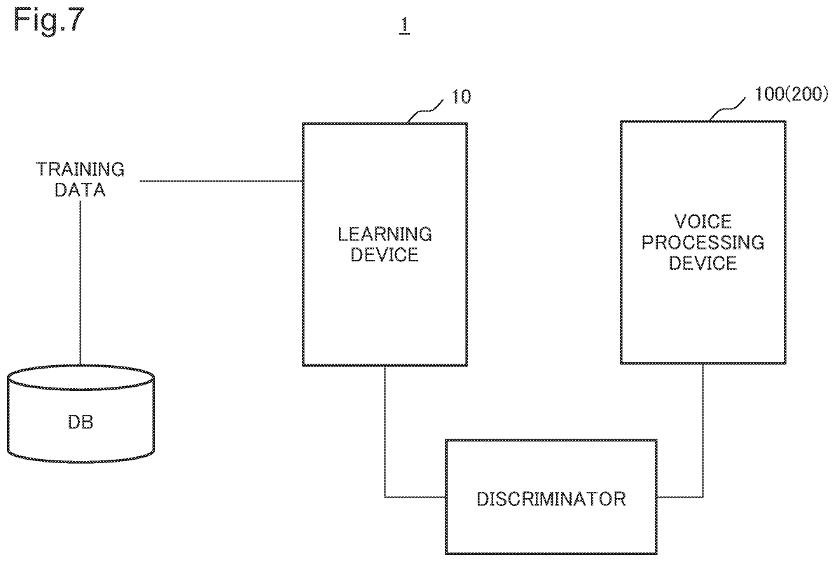

The feature extraction unit 110 extracts, from input data based on an utterance of a person to be determined, a feature of the input data using a discriminator (FIG. 1 or 7) that has performed machine learning using, as training data, voice data based on an utterance of the person to be determined in a normal state. The feature extraction unit 110 is an example of a feature extraction means. The training data is voice data based on an utterance of the person to be determined in a normal state.

In one example, the feature extraction unit 110 receives input data (FIG. 1) input using an input device such as a microphone. In addition, the feature extraction unit 110 receives registered data (FIG. 1) from a DB, which is not illustrated. The feature extraction unit 110 inputs the input data to a trained discriminator (hereinafter simply referred to as a discriminator, and extracts a feature of the input data from the discriminator. In addition, the feature extraction unit 110 inputs the registered data to the discriminator, and extracts a feature of the registered data from the feature extraction unit 110.

The feature extraction unit 110 may use any machine learning method in order to extract the respective features of the input data and the registered data. Here, an example of the machine learning is deep learning, and an example of the discriminator is a deep neural network (DNN). In this case, the feature extraction unit 110 inputs input data to the DNN, and extracts a feature of the input data from an intermediate layer of the DNN. In one example, the feature extracted from the input data may be a mel-frequency cepstrum coefficient (MFCC) or a linear predictive coding (LPC) coefficient, or may be a power spectrum or a spectral envelope. Alternatively, the feature of the input data may be a certain-dimensional feature vector including a feature amount obtained by frequency-analyzing voice data (hereinafter referred to as an acoustic vector).

The feature extraction unit 110 outputs data on the feature of the registered data and data on the feature of the input data to the index value calculation unit 120.

The index value calculation unit 120 calculates an index value indicating a degree of similarity between the feature of the input data and the feature of the voice data based on the utterance of the person to be determined in the normal state. The index value calculation unit 120 is an example of an index value calculation means. The voice data based on the utterance of the person to be determined in the normal state corresponds to the registered data described above.

In one example, the index value calculation unit 120 receives the data on the feature of the input data from the feature extraction unit 110. In addition, the index value calculation unit 120 receives the data on the feature of the registered data from the feature extraction unit 110. The index value calculation unit 120 discriminates each of phonemes included in the input data and phonemes included in the registered data. The index value calculation unit 120 associates the phonemes included in the input data with the same phonemes included in the registered data.

Next, in one example, the index value calculation unit 120 calculates scores indicating degrees of similarity between features of the phonemes included in the input data and features of the same phonemes included in the registered data, respectively, and calculates the sum of the scores calculated for all the phonemes as an index value. The feature of the phoneme included in the input data and the feature of the phoneme included in the registered data may be feature vectors in the same dimension. In addition, the score indicating a degree of similarity may be an inverse number of a distance between the feature vector of the phoneme included in the input data and the feature vector of the same phoneme included in the registered data, or “(upper limit value of distance)-distance”. Note that, in the following description, the “score” refers to the sum of the scores described above. In addition, the “feature of the input data” and the “feature of the registered data” refer to a “feature of a phoneme included in the input data” and a “feature of the same phoneme included in the registered data”, respectively.

The index value calculation unit 120 outputs data on the calculated index value (the score in one example) to the state determination unit 130.

The state determination unit 130 determines whether the person to be determined is in a normal state or in an unusual state based on the index value. The state determination unit 130 is an example of a state determination means. In one example, the state determination unit 130 receives, from the index value calculation unit 120, data on the index value indicating a degree of similarity between the feature of the input data and the feature of the registered data.

Next, in one example, the state determination unit 130 compares the index value with a predetermined threshold value. When the index value is larger than the threshold value, the state determination unit 130 determines that the person to be determined is in a normal state. On the other hand, when the index value is equal to or smaller than the threshold value, the state determination unit 130 determines that the person to be determined is in an unusual state. The state determination unit 130 outputs a determination result.

In addition, the state determination unit 130 may restrict an authority of the person to be determined to operate an object. For example, the object is a commercial vehicle to be operated by the person to be determined. In this case, the state determination unit 130 may control a computer of the commercial vehicle not to start an engine of the commercial vehicle.

(Operation of Voice Processing Device 100)

An example of the operation of the voice processing device 100 according to the second example embodiment will be described with reference to FIG. 3. FIG. 3 is a flowchart illustrating a flow of processes executed by each unit (FIG. 2) of the voice processing device 100 in the present example.

As illustrated in FIG. 3, the feature extraction unit 110 extracts a feature of input data (FIG. 1) from the input data (S101). In addition, the feature extraction unit 110 extracts a feature of registered data (FIG. 1) from the registered data. Then, the feature extraction unit 110 outputs data on the feature of the input data and data on the feature of the registered data to the index value calculation unit 120.

The index value calculation unit 120 receives the data on the feature of the input data and the data on the feature of the registered data from the feature extraction unit 110. The index value calculation unit 120 calculates an index value indicating a degree of similarity between the feature of the input data and the feature of the registered data (S102). In one example, the index value calculation unit 120 calculates, as an index value, a score indicating a distance between a feature vector indicating the feature of the input data and a feature vector indicating the feature of the registered data. The index value calculation unit 120 outputs data on the calculated index value (score) to the state determination unit 130.

The state determination unit 130 receives, from the index value calculation unit 120, data on the score indicating a degree of similarity between the feature of the input data and the feature of the registered data. The state determination unit 130 compares the score with a predetermined threshold value (S103).

When the score is larger than the threshold value (Yes in S103), the state determination unit 130 determines that the person to be determined is in a normal state (S104A).

On the other hand, when the score is equal to or smaller than the threshold value (No in S103), the state determination unit 130 determines that the person to be determined is in an unusual state (S104B). Thereafter, the state determination unit 130 may output a determination result (step S104A or S104B).

Then, the operation of the voice processing device 100 according to the second example embodiment ends.

### Effects of Present Example Embodiment

According to the configuration of the present example embodiment, the feature extraction unit 110 extracts, from input data based on an utterance of a person to be determined, a feature of the input data using a discriminator that has performed machine learning using, as training data, voice data based on an utterance of the person to be determined in a normal state. The index value calculation unit 120 calculates an index value indicating a degree of similarity between the feature of the input data and the feature of the voice data based on the utterance of the person to be determined in the normal state. The state determination unit 130 determines whether the person to be determined is in a normal state or in an unusual state based on the index value. The voice processing device 100 can acquire an index value indicating a probability that the person is in a normal state using the discriminator. A determination result based on the index value indicates how similar an utterance of the person to be determined is to the utterance of the person in the normal state. Therefore, the voice processing device 100 is capable of easily determining a state (a normal state or an unusual state) of the person to be determined, without requiring a user to conduct an interview with the person to be determined or without requiring a biological sensor. Furthermore, in a case where the result of the determination made by the voice processing device 200 is output, the user can immediately check the state of the person to be determined.

### Third Example Embodiment

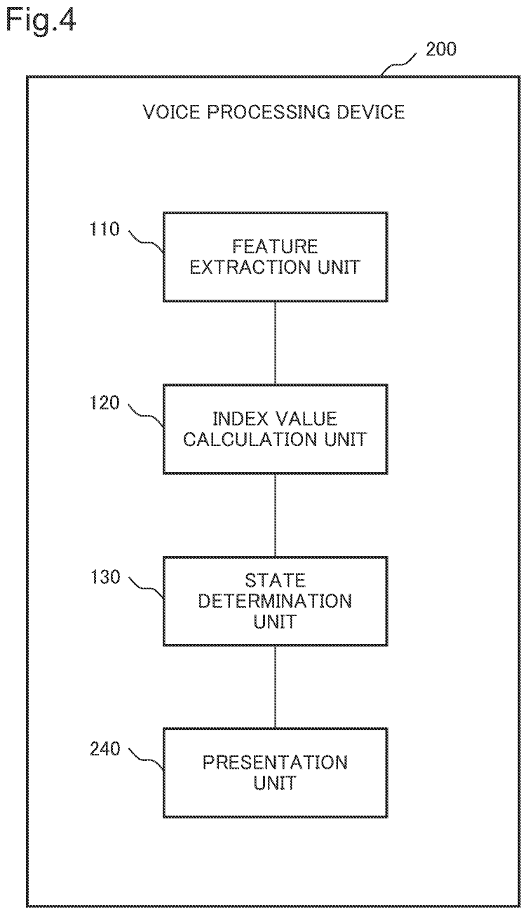

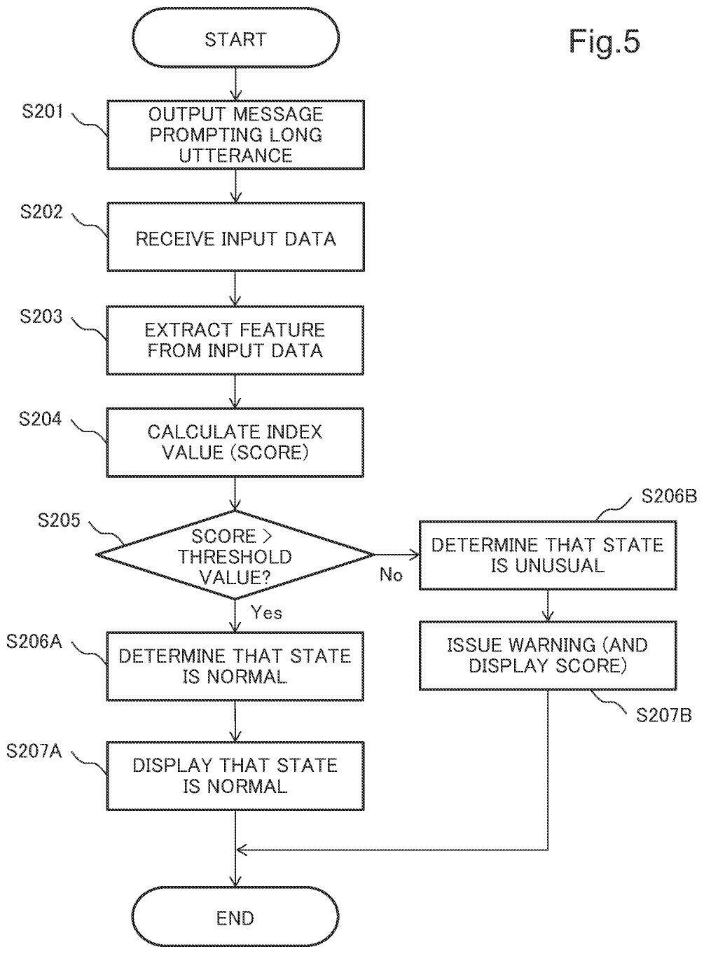

A third example embodiment will be described with reference to FIGS. 4 and 5.

(Voice Processing Device 200)

An outline of an operation of a voice processing device 200 according to the third example embodiment is common to the operation of the voice processing device 100 described above in the second example embodiment. Basically, the voice processing device 200 operates in common with the voice processing device X00 described with reference to FIG. 1 in the first example embodiment, but also operates in a partially different manner from the voice processing device X00 as will be described below.

FIG. 4 is a block diagram illustrating a configuration of the voice processing device 200 according to the third example embodiment. As illustrated in FIG. 4, the voice processing device 200 includes a feature extraction unit 110, an index value calculation unit 120, and a state determination unit 130. In addition, the voice processing device 200 further includes a presentation unit 240. That is, the configuration of the voice processing device 200 according to the third example embodiment is different from that of the voice processing device 100 according to the second example embodiment in that the presentation unit 240 is included. In the third example embodiment, the processes performed by the components denoted by the same reference signs as those in the second example embodiment are common. Therefore, in the third example embodiment, only the process performed by the presentation unit 240 will be described.

The presentation unit 240 presents information indicating whether a person to be determined is in a normal state or in an unusual state based on a result of a determination made by the state determination unit 130 of the voice processing device 200. The presentation unit 240 is an example of a presentation means.

In one example, the presentation unit 240 acquires data on the determination result indicating whether the person to be determined is in a normal state or in an unusual state from the state determination unit 130. The presentation unit 240 may present different information depending on the data on the determination result.

For example, when the state determination unit 130 determines that the person to be determined is in a normal state, the presentation unit 240 acquires data on the index value (score) calculated by the index value calculation unit 120, and presents information indicating a probability of the determination result based on the index value (score). Specifically, the presentation unit 240 displays that the person to be determined is in a normal state on the screen using text, a symbol, or light. On the other hand, when the state determination unit 130 determines that the person to be determined is in an unusual state, the presentation unit 240 issues a warning. In addition, the presentation unit 240 may acquire data on the index value (score) calculated by the index value calculation unit 120, and output the acquired data on the index value (score) to a display device, which is not illustrated, to display the index value (score) on a screen of the display device.

### Operation of Voice Processing Device 200

An operation of the voice processing device 200 according to the third example embodiment will be described with reference to FIG. 5. FIG. 5 is a flowchart illustrating processes executed by each unit (FIG. 4) of the voice processing device 200.

As illustrated in FIG. 5, the presentation unit 240 outputs data on a message prompting to the person to be determined to give a long utterance to the display device, which is not illustrated, so that the message is displayed on the screen of the display device (S201). The user of the voice processing device 200 may appropriately determine the meaning of the long utterance (or the definition of the length of the utterance). In one example, the long utterance is an utterance including N or more words (N is the number set by the user). The reason why the person to be determined is required to give a long utterance is to accurately calculate an index value indicating a degree of similarity between the feature of the input data and the feature of the registered data.

The feature extraction unit 110 receives, from an input device such as a microphone, a voice signal (input data in FIG. 1) obtained by collecting sound from the utterance of the person to be determined (S202). In addition, the feature extraction unit 110 receives, from the DB, a voice signal (registered data in FIG. 1) recorded when the person to be determined is in a normal state.

The feature extraction unit 110 extracts a feature of the input data from the input data (S203). In addition, the feature extraction unit 110 extracts a feature of the registered data from the registered data.

Then, the index value calculation unit 120 calculates an index value (score) indicating a degree of similarity between the feature of the input data and the feature of the registered data (S204).

The state determination unit 130 compares the index value with a predetermined threshold value (S205). When the score is larger than the threshold value (Yes in S205), the state determination unit 130 determines that the person to be determined is in a normal state (S206A). The state determination unit 130 outputs a determination result to the presentation unit 240. In this case, the presentation unit 240 displays information indicating that the person to be determined is in a normal state on a display device, which is not illustrated (S207A).

On the other hand, when the score is equal to or smaller than the threshold value (No in S205), the state determination unit 130 determines that the person to be determined is in an unusual state (S206B). The state determination unit 130 outputs a determination result to the presentation unit 240. In this case, the presentation unit 240 issues a warning (S207B).

In addition, in step S207B, the presentation unit 240 may display information indicating that the person to be determined is in an unusual state on the display device, which is not illustrated. In one example, the presentation unit 240 acquires data on the index value (score) calculated by the index value calculation unit 120 in step S204, and displays the acquired score itself or information based on the score (in one example, a suggestion of a retest) on the display device.

Then, the operation of the voice processing device 200 according to the third example embodiment ends.

### Effects of Present Example Embodiment

According to the configuration of the present example embodiment, the feature extraction unit 110 extracts, from input data based on an utterance of a person to be determined, a feature of the input data using a discriminator that has performed machine learning using, as training data, voice data based on an utterance of the person to be determined in a normal state. The index value calculation unit 120 calculates an index value indicating a degree of similarity between the feature of the input data and the feature of the voice data based on the utterance of the person to be determined in the normal state. The state determination unit 130 determines whether the person to be determined is in a normal state or in an unusual state based on the index value. As a result, the voice processing device 200 can acquire an index value indicating a probability that the person to be determined is in a normal state using the discriminator. A determination result based on the index value indicates how similar an utterance of the person to be determined is to the utterance of the person in the normal state. Therefore, the voice processing device 200 is capable of easily determining a state (a normal state or an unusual state) of the person to be determined, without a result of an interview conducted by a user with the person to be determined or without requiring biological data. Furthermore, in a case where the result of the determination made by the voice processing device 200 is output, the user can immediately check the state of the person to be determined.

Furthermore, according to the configuration of the present example embodiment, the presentation unit 240 presents information indicating whether the person to be determined is in a normal state or in an unusual state based on the determination result. Therefore, the user can easily ascertain the state of the person to be determined by seeing the presented information. Then, the user can perform an appropriate measure (e.g., a re-interview with a crew member or a restriction of work) according to the ascertained state of the person to be determined.

[Hardware Configuration]

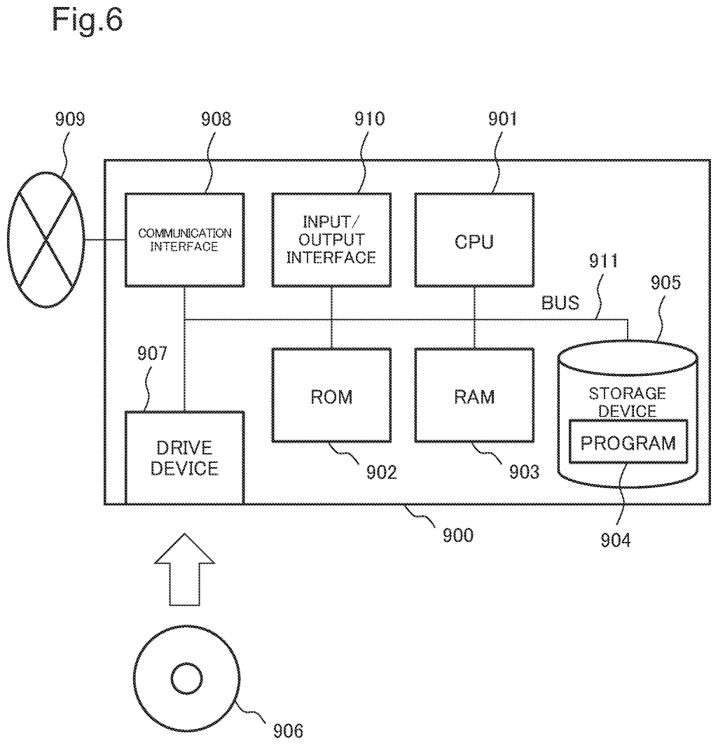

Each of the components of the voice processing devices 100 and 200 described in the second and third example embodiments represents a functional unit block. Some or all of these components are implemented, for example, by an information processing apparatus 900 as illustrated in FIG. 6. FIG. 6 is a block diagram illustrating an example of a hardware configuration of the information processing apparatus 900.

As illustrated in FIG. 6, the information processing apparatus 900 includes the following components as an example.
Central Processing Unit (CPU) 901
Read Only Memory (ROM) 902
Random Access Memory (RAM) 903
Program 904 loaded into the RAM 903
Storage device 905 storing the program 904
Drive device 907 reading and writing a recording medium 906
Communication interface 908 connected to a communication network 909
Input/output interface 910 for inputting/outputting data
Bus 911 connecting the components to each other

The components of the voice processing devices 100 and 200 described in the second and third example embodiments are implemented by the CPU 901 reading and executing the program 904 for implementing their functions. The program 904 for implementing the functions of the components is stored, for example, in the storage device 905 or the ROM 902 in advance, and the CPU 901 loads the program 904 into the RAM 903 for execution if necessary. Note that the program 904 may be supplied to the CPU 901 via the communication network 909, or may be stored in advance in the recording medium 906 such that the drive device 907 reads the program to be supplied to the CPU 901.

According to the above-described configuration, each of the voice processing devices 100 and 200 described in the second and third example embodiments is implemented as hardware. Therefore, effects similar to those described in the second and third example embodiments can be obtained.

### Common to Second and Third Example Embodiments

An example of a configuration of a voice authentication system to which the voice processing device according to the second or third example embodiment is commonly applied will be described.

(Voice Authentication System 1)

An example of a configuration of a voice authentication system 1 will be described with reference to FIG. 7. FIG. 7 is a block diagram illustrating an example of a configuration of a voice authentication system 1.

As illustrated in FIG. 7, the voice authentication system 1 includes a voice processing device 100(200) and a learning device 10. Further, the voice authentication system 1 may include one or more input devices. The voice processing device 100(200) is the voice processing device 100 according to the second example embodiment or the voice processing device 200 according to the third example embodiment.

As illustrated in FIG. 7, the learning device 10 acquires training data from a data base (DB) on a network or from a DB connected to the learning device 10. The learning device 10 trains the discriminator using the acquired training data. More specifically, the learning device inputs voice data included in the training data to the discriminator, gives correct answer information included in the training data to an output of the discriminator, and calculates a value of a loss function, which has been known. Then, the learning device 10 repeatedly updates a parameter of the discriminator over a predetermined number of times to reduce the calculated value of the loss function. Alternatively, the learning device 10 repeatedly updates a parameter of the discriminator until the value of the loss function becomes equal to or smaller than a predetermined value.

As described in the second example embodiment, the voice processing device 100 determines a state of a person to be determined using the trained discriminator. Similarly, the voice processing device 200 according to the third example embodiment also determines a state of a person to be determined using the trained discriminator.

### INDUSTRIAL APPLICABILITY

In one example, the present disclosure can be used in a voice authentication system that identifies a person by analyzing voice data input using an input device.

### REFERENCE SIGNS LIST

1 voice authentication system
10 learning device
100 voice processing device
110 feature extraction unit
120 index value calculation unit
130 state determination unit
200 voice processing device
240 presentation unit

What is claimed is:

1. A voice processing device comprising:
a memory configured to store instructions; and
at least one processor configured to execute the instructions to perform;
extracting, from input data based on an utterance of a person to be determined, a first feature of the input data using a discriminator that has performed machine learning using, as training data, voice data based on an utterance of the person to be determined in a normal state;
calculating an index value indicating a degree of similarity between the first feature of the input data and a second feature of the voice data based on the utterance of the person to be determined in the normal state; and
determining whether the person to be determined is in the normal state or in an unusual state based on the index value.

2. The voice processing device according to claim 1, wherein
the at least one processor is configured to execute the instructions to perform;
presenting information indicating whether the person to be determined is in the normal state or in the unusual state based on a result of the determination.

3. The voice processing device according to claim 2, wherein
when it is determined that the person to be determined is in an unusual state,
the at least one processor is configured to execute the instructions to perform;
presenting information indicating a probability of the result of the determination based on the index value.

4. The voice processing device according to claim 1, wherein
when it is determined that the person to be determined is in an unusual state,
the at least one processor is configured to execute the instructions to perform;
restricting an authority of the person to be determined to operate an object.

5. A voice processing method comprising:
extracting, from input data based on an utterance of a person to be determined, a first feature of the input data using a discriminator that has performed machine learning using, as training data, voice data based on an utterance of the person to be determined in a normal state;
calculating an index value indicating a degree of similarity between the first feature of the input data and a second feature of the voice data based on the utterance of the person to be determined in the normal state; and
determining whether the person to be determined is in the normal state or in an unusual state based on the index value.

6. A non-transitory recording medium storing a program for causing a computer to execute:
extracting, from input data based on an utterance of a person to be determined, a first feature of the input data using a discriminator that has performed machine learning using, as training data, voice data based on an utterance of the person to be determined in a normal state;
calculating an index value indicating a degree of similarity between the first feature of the input data and a second feature of the voice data based on the utterance of the person to be determined in the normal state; and
determining whether the person to be determined is in the normal state or in an unusual state based on the index value.

7. (canceled)
